(19) United States
(12) Patent Application Publication
(10) Pub. No.: US 2024/0158573 A1
(43) Pub. Date: May 16, 2024

(54) POLYHYDROXYALKANOATE COMPOSITIONS AND METHODS OF MAKING THE SAME

(71) Applicant: PHAXTEC, INC., WAKE FOREST, NC (US)

(72) Inventors: Robert Keith SALSMAN, Hoschton, GA (US); Anindya MUKHERJEE, WAKE FOREST, NC (US); Nicolas George TRIANTAFILOPOULOS, SAN DIEGO, CA (US)

(21) Appl. No.: 18/552,038

(22) PCT Filed: Mar. 25, 2022
(86) PCT No.: PCT/US22/21982

§ 371 Date: Sep. 22, 2023

Related U.S. Application Data
(60) Provisional application No. 63/166,031, filed on Mar. 25, 2021.

(51) Int. Cl. C08G 63/81; C08G 63/06

(52) U.S. Cl. CPC C08G 63/81; C08G 63/06

(57) ABSTRACT

In alternative embodiments, provided are PHA compositions comprising a polyhydroxyalkanoate (PHA) polymer and a viscosity modifying agent. The composition can also include a nucleating agent, an antioxidant, a dispersant, a viscosity modifying agent, an ultraviolet light inhibitor, an emulsifying agent and/or a stabilizer. Methods of manufacturing include forming the PHA composition into a polymer melt or an aqueous dispersion. The PHA composition can be used as a coating on paper, cardboard or paperboard substrates, or other surfaces, including on inks or adhesives.

### RELATED APPLICATIONS

This Patent Convention Treaty (PCT) International Application claims the benefit of priority under 35 U.S.C. § 119(e) of U.S. Provisional Application No. 63/166,031, Mar. 25, 2021. The aforementioned application is expressly incorporated herein by reference in its entirety and for all purposes. All publications, patents, patent applications cited herein are hereby expressly incorporated by reference for all purposes.

### TECHNICAL FIELD

This invention generally relates to polymers and their formulations. In alternative embodiments, provided are compositions comprising polyhydroxyyalkanoates (PHAs), and methods for making such compositions, and their use in products of manufacture such as coatings for paper, paperboard, cardboard, cellulose fiber-comprising packaging materials, or in other products such as coatings, inks, and adhesives.

### BACKGROUND

Polyhydroxyalkanoates (PHAs) are carbon-storage polymers produced by a variety of natural microorganisms. Changes in nutrient availability trigger production of PHAs in PHA-producing microorganisms, which the organisms accumulate intracellularly in lipid walled sacs. Accordingly, PHAs are naturally produced and biodegradable in soil, fresh water, and marine environments, where PHA-consuming microorganisms exist. Hence, PHAs are compostable in several environments such as home, gardens or industrial composting complexes.

PHA becomes semicrystalline when processed under heat and can act as a thermoplastic or a resin thus making them useful in replacing many of the world's plastics, paints, coatings, inks, adhesives and as medical devices for in vivo use such as sutures, mesh and as parts of organs such as stomach walls. PHAs are non-toxic and safe. The complete breakdown of PHA materials into CH₄, CO₂, water, and minerals can be achieved in home and garden composters, conventional industrial composters, or anaerobic digesters.

Different monomers can be combined to form PHAs to give materials with different properties. Therefore, PHAs can have melting points ranging from 40 to 180° C. The mechanical properties and biocompatibility of PHAs can be changed by blending, modifying their surface, or combining PHAs with other polymers, enzymes, and inorganic materials which makes it possible to use in a wide range of end-use applications. Moreover, the combination of plastic-like properties, superior end-of-life properties, and biodegradability make PHAs beneficial as both single use plastics as well in durable applications. They can be spun into fibers for use as nonwovens in diapers, tissue, and fabrics and used as coatings on paper and paperboard products.

Coatings on paper and paperboard products is of particular interest because paper fiber-based products are recyclable and PHAs barrier coatings, replacing polyethylene (PE), can be a candidate for sustainable packaging, contributing to the circular economy by recycling or composting single-use foodservice packaging, a major waste pollutant globally. Although many different chemistries have been attempting to economically replace PE, PHA polymeric coatings have the advantages of (a) providing barrier performance, (b) being able, with modifications, to be applied on existing equipment, and (c) give the final product recyclability and compostability under all environments (i.e., industrial, curbside, marine). Therefore, there is interest to develop cost-effective PHA-based coating formulations that impart water moisture, grease and oil, as well as oxygen barrier to paper and paperboard for packaging.

PHAs are envisioned as a sustainable solution for future polymer manufacturing. Previous attempts to produce PHAs from carbohydrates have been hindered by high carbon feedstock costs and the low cost of competing petroleum-based and non-biodegradable polymers. A PHA composition that overcomes the deficiencies discussed above is desired. Additionally, a cost-effective solution for PHA production and PHA film production is also desired.

### SUMMARY

In alternative embodiments, provided are products of manufacture and compositions comprising a polyhydroxyalkanoate (PHA) polymer, or copolymer, and a viscosity modifying agent, and other additives, to generate or formulate aqueous dispersions, which can be used for example as a barrier paper, cellulose fiber-comprising packaging materials and/or for paperboard or cardboard coatings.

In alternative embodiments, the viscosity modifying agent comprises an ester.

In alternative embodiments, the PHA polymer comprises one or more of 3-hydroxybutyrate (3HB) homopolymer, or a copolymer of 3-hydroxybutyrate (3HB) and 4-hydroxybutyrate (4HB), or a copolymer of 3-hydroxybutyrate (3HB) and 3-hydroxyvalerate (3HV), or a copolymer of 3-hydroxybutyrate (3HB) and 3-hydroxyhexanoate (3H) polymers.

In an alternative aspect, a PHA composition as provided herein comprises a copolymer of PHA comprising 8% to 30% 41113 and 92% to 70% 3HB, and an ester with a melting point above 30° C., or above 40° C., or over 50° C., or an ester with a melting point between about 25° C. and 75° C.

In alternative embodiments, the composition or product of manufacture further comprises at least one (or one or two or several) nucleating agent(s), which can comprise a poly 3-hydroxybutyrate (P3-HB), or a P3-HB powder, wherein optionally the P3-HB or P3-HB powder comprises between about 0.05% to 5%, or 0.05% to 10%, or 0.1% to 3%, or 0.1% to 2% by volume, or about 0.05%, 1%, 2%, 3%, 4% or 5% by volume.

In alternative embodiments, the nucleating agent or agents comprise one, two or several of: sulfur; an erythritol; pentaerythritol; dipentaerythritol; a stearate; sorbitol; mannitol; a polyester wax; a sebacate; a citrate; a fatty ester of adipic, succinic, and/or glucaric acid; a lactate; an alkyl diester; a citrate or citrates; an alkyl methyl esters; a dibenzoate; a clay or clays such as a nano clay; calcium carbonate or talc; kaolinite; mont-morillonite; bentonite; silica; chitin; titanium dioxide; mica; propylene carbonate; a caprolactone diol; a poly(ethylene) glycol (PEG); an ester of a vegetable oils; a long chain alkyl acids; an adipate; glycerol; an isosorbide or isosorbide derivative; and, mixtures thereof.

In alternative embodiments, the nucleating agent or agents comprise between about 0.01% to 5%, or 0.1% to 4%, or 0.5% to 3%, of the volume of the mixture, or between about 0.05% to 10%, or 0.1% to 3%, or 0.1% to 2% by volume, or about 0.05%, 1%, 2%, 3%, 4% or 5% by volume or by weight.

In alternative embodiments, the nucleating agent or agents comprise propylene carbonate or caprolactone diol, optionally having a number average molecular weight (MW) from between about 1,000 to 4,000 g/mol, or a poly(ethylene) glycols having a number average MW of between about 1,000 to 4,000 g/mol, of an ester or esters of vegetable oils, long chain alkyl acids, adipates, glycerol, isosorbide derivatives or mixtures thereof.

In alternative embodiments, a PHA composition as provided herein comprises: a copolymer of PHA comprising between about 8% to 24% 4-HB (or between about 6% to 30% 4-HB, or between about 4% to 40% 4-HB) and between about 92% to 76% 3-HB (or between about 96% to 60% 3-HB); and, an ester having a melting point above about 30° C., or above about 50° C., or between about 30° C. and 50° C. or 25° C. and 75° C.; and optionally the PHA composition further comprises a nucleating agent, wherein optionally the nucleating agent is (or comprises) a P3-HB powder.

In alternative embodiments, provided are compositions comprising a polyhydroxyalkanoate (PHA) polymer and a viscosity modifying agent; and optionally the viscosity modifying agent comprises an ester (optionally a fatty acid ester), and optionally the viscosity modifying agent comprises, or further comprises, glycerin, trimethyl propanol (TMP), pentaerythritol, and/or sorbitol.

In alternative embodiments of compositions as provided herein:
the ester comprises a fatty acid ester of a polyhydroxy alcohol, and optionally the fatty acid ester comprises lauric acid, myristic acid, palmitic acid, stearic acid, arachidic acid, or behenic acid;
the fatty acid ester has a melting point above about 50° C., or between about 50° C. and 80° C.;
the PHA polymer comprises a PHA copolymer, and optionally the PHA polymer or the PHA copolymer comprises one or more of 3-hydroxybutyrate (3HB) homopolymer, or a copolymer of 3-hydroxybutyrate (3HB) and 4-hydroxybutyrate (4HB), or a copolymer of 3-hydroxybutyrate (3HB) and 3-hydroxyvalerate (3HV), or a copolymer of 3-hydroxybutyrate (3HB) and 3-hydroxyhexanoate (3HH) polymers;
the PHA polymer or the PHA copolymer comprises 8% to 30% 41113 and 92% to 70% 3HB, and an ester with a melting point above 30° C., or above 40° C., or over 50° C., or an ester with a melting point between about 25° C. and 75° C.;
the PHA polymer comprises one or more of 3-hydroxybutyrate or 4-hydroxybutyrate;
the PHA polymer, or the composition, has a non-tack coating time of less than about 30 seconds, or less than about 10 seconds, or less than about 1 second (sec);
the viscosity modifying agent is present in an amount of up to about 15 wt %, or between about 0.5% and 20 wt %, or between about 1% and 15 wt %, or optionally the viscosity modifying agent is present in an amount of about 5 wt % to about 15 wt %, about 7 wt % to about 13 wt %, or about 8 wt % to about 12 wt %;
the composition further comprises a nucleating agent, wherein optionally the nucleating agent or agents comprise between about 0.01% to 5%, or 0.1% to 4%, or 0.5% to 3%, of the volume of the PHA composition, or between about 0.05% to 10%, or 0.1% to 3%, or 0.1% to 2% by volume, or about 0.05%, 1%, 2%, 3%, 4% or 5% by volume of the PHA composition, and optionally the nucleating agent comprises one, two or several of: sulfur; an erythritol; pentaerythritol; dipentaerythritol; a stearate; sorbitol; mannitol; a polyester wax; a sebacate; a citrate; a fatty ester of adipic, succinic, and/or glucaric acid; a lactate; an alkyl diester; a citrate or citrates; an alkyl methyl esters; a dibenzoate; a clay or clays or a nano clay; calcium carbonate or talc; kaolinite; mont-morillonite; bentonite; silica; chitin; titanium dioxide; mica; propylene carbonate; a caprolactone diol; a poly(ethylene) glycol (PEG); an ester of a vegetable oils; a long chain alkyl acids; an adipate; glycerol; an isosorbide or isosorbide derivative; and, mixtures thereof, and optionally the nucleating agent or agents comprise propylene carbonate or caprolactone diol, optionally having a number average molecular weight (MW) from between about 1,000 to 4,000 g/mol, or a poly(ethylene) glycols having a number average MW of between about 1,000 to 4,000 g/mol, of an ester or esters of vegetable oils, long chain alkyl acids, adipates, glycerol, isosorbide derivatives or mixtures thereof,
the nucleating agent comprises poly-3-hydroxy butyrate (P3-HB), and optionally the P3-HB comprises or is formulated as a P3-HB powder or powder melt, and optionally the nucleating agent comprises precipitated calcium carbonate, precipitated or fumed silica, talc, bentonite or montmorillonite clay, calcium sulfate and/or boron nitride, and optionally the size of the P3-HB powder is less than about 1000 microns, or less than about 100 microns, or less than about 40 microns, or between about 20 microns and 1000 microns; and/or
the composition further comprises:
   (a) at least one antioxidant, wherein optionally the antioxidant comprises sorbic acid, butylated hydroxytoluene (BHT), butylated hydroxyanisole (BHA) or a combination thereof,
   (b) an antimicrobial, wherein optionally the antimicrobial comprises BHT, BHA, benzoic acid, ascorbic acid or a combination thereof;
   (c) a dispersant, wherein optionally the dispersant comprises an ester of an unsaturated acid or a polyacrylic acid or a combination thereof;
   (d) a thickener or a viscosity modifier, wherein optionally the thickener or the viscosity modifier comprises glycerin mono stearate (GMS), an alginate, a polyvinyl alcohol or a combination thereof;
   (e) an ultraviolet light inhibitor, wherein optionally the ultraviolet light inhibitor comprises a hindered amine light stabilizers, a stearate of magnesium or zinc, or a combination thereof,
   (f) a stabilizer, wherein optionally a heat stabilizer, wherein optionally the stabilizer comprises polyvinyl alcohol (PVOH), vinyl alcohol, a soaps of a fatty acid, pyrrolidone, an ethylene oxide, a propylene oxide or a combination thereof,
   (g) an emulsifying agent, wherein optionally the emulsifying agent comprises a polyethylene oxide addict a of fatty acid and fatty alcohol, or a polysorbate;
   (h) an anti-blocking agent, wherein optionally the anti-blocking agent comprises: an inorganic mineral pigment; a diatomaceous earth; talc; sodium or potassium aluminosilicate; a mineral mixture of sodium and potassium aluminosilicates; or, a silica, optionally a synthetic, amorphous silica; or
   (i) any combination of (a) to (h).

In alternative embodiments, provided are composite structures or products of manufacture comprising a composition as provided herein.

In alternative embodiments, provided are composite structures or products of manufacture comprising a substrate and a coating comprising a composition as provided herein.

In alternative embodiments or the composite structures or products of manufacture:
the substrate comprises a paper or a paperboard stock, or a cellulose fiber-comprising packaging material; and/or
the coating has a thickness of about 5 millimeters (mm), or between about 0.25 to 20 mm, or between about 0.5 to 20 mm, or between about 1 to 10 mm.

In alternative embodiments, provided are methods of producing a polyhydroxyalkanoate (PHA) polymer powder comprising: steps as set forth in FIG. 1, or the following steps:
(a) adding a substrate, a PHA-producing microorganism (optionally a methanotrophic microorganism), micronutrients and oxygen to a culture vessel or a fermentation reaction vessel or equivalent;
(b) culturing the microorganism in the culture vessel or the fermentation reaction vessel or equivalent;
(c) separating the PHA from the cultured microorganism, optionally the separate comprises filtration or centrifugation;
(d) suspending the PHA in water, optionally using purified or distilled water;
(e) centrifuging and/or filtering the PHA suspension to remove any remaining non-PHA debris from the microorganism to generate a purified PHA; and
(f) spray-drying the purified PHA to generate a PHA powder.

In alternative embodiments, provided are methods of producing a polyhydroxyalkanoate (PHA) composition or dispersion comprising: steps as set forth in FIG. 2 and FIG. 3, or the following steps:
(a) inputting or injecting a PHA pellet or powder into a polymer jet micronizer, thereby producing micronized PHA powder;
(b) adding the micronized PHA powder, optionally with at least one additive, and water into a reaction vessels to generate a PHA aqueous solution or a PHA suspension, wherein optionally water comprises about 50% to 60% of the volume of the aqueous solution or a PHA suspension,
and optionally the at least one additive comprises a nucleating agent or a plasticizer; and
(c) adding the PHA aqueous or water-based solution or the PHA suspension to a high shear mixer to generate a PHA dispersion,
and optionally a step (d) packaging the PHA aqueous or water-based dispersion, optionally into totes.

In alternative embodiments of methods as provided herein, the additive comprises:
(a) at least one antioxidant, wherein optionally the antioxidant comprises sorbic acid, butylated hydroxytoluene (BHT), butylated hydroxyanisole (BHA) or a combination thereof,
(b) an antimicrobial, wherein optionally the antimicrobial comprises BHT, BHA, benzoic acid, ascorbic acid or a combination thereof;
(c) a dispersant, wherein optionally the dispersant comprises an ester of an unsaturated acid or a polyacrylic acid or a combination thereof;
(d) a thickener or a viscosity modifier, wherein optionally the thickener or the viscosity modifier comprises glycerin mono stearate (GMS), an alginate, a polyvinyl alcohol or a combination thereof;
(e) an ultraviolet light inhibitor, wherein optionally the ultraviolet light inhibitor comprises a hindered amine light stabilizers, a stearate of magnesium or zinc, or a combination thereof,
(f) a stabilizer, wherein optionally a heat stabilizer, wherein optionally the stabilizer comprises polyvinyl alcohol (PVOH), vinyl alcohol, a soaps of a fatty acid, pyrrolidone, an ethylene oxide, a propylene oxide or a combination thereof,
(g) an emulsifying agent, wherein optionally the emulsifying agent comprises a polyethylene oxide addict a of fatty acid and fatty alcohol, or a polysorbate;
(h) an anti-blocking agent, wherein optionally the anti-blocking agent comprises: an inorganic mineral pigment; a diatomaceous earth; talc; sodium or potassium aluminosilicate; a mineral mixture of sodium and potassium aluminosilicates; or, a silica, optionally a synthetic, amorphous silica;
(i) a viscosity modifying agent, wherein optionally the viscosity modifying agent comprises an ester, or optionally the viscosity modifying agent comprises glycerin, trimethyl propanol (TMP), pentaerythritol, and/or sorbitol; or
(j) any combination of (a) to (i).

In alternative embodiments, provided are methods of producing a polyhydroxyalkanoate (PHA) polymer melt comprising: steps as set forth in FIG. 4, or the following steps:
(a) melting a PHA powder to generate a melt extrusion; and
(b) pelletilizing the PHA melt extrusion, optionally pelletilizing by using an extruder,
and optionally a step (c) packaging the PHA extrusion, wherein optionally the PHA extrusion is fabricated as a pellet or a powder, and optionally packaging into totes or a container.

In alternative embodiments, provided are polyhydroxyalkanoate (PHA) compositions comprising:
(a) a copolymer of PHA comprising
   (i) between about 8% to 24% 4-HB and
   (ii) between about 92% to 76% 3-HB and
(b) an ester having a melting point above about 30° C., or above 50° C., or having a melting point between about 30° C. and 75° C., or having a melting point between about 40° C. and 60° C.

In alternative embodiments of PHA compositions as provided herein:
the PHA copolymer comprises about 16% 4-HB and about 84% 3-HBk;
the ester is a reacted product of a saturated fatty acid and a polyhydroxy alcohol;
the PHA composition is formulated or configured for application to a paper, cardboard, or a paperboard stock, or a resin or a plastic, as a barrier coating;
the PHA composition further comprises a nucleating agent, wherein optionally the nucleating agent or agents comprise between about 0.01% to 5%, or 0.1% to 4%, or 0.5% to 3%, of the volume of the PHA composition, or between about 0.05% to 10%, or 0.1% to 3%, or 0.1% to 2% by volume, or about 0.05%, 1%, 2%, 3%, 4% or 5% by volume of the PHA composition, and optionally the nucleating agent comprises a P3-HB, and optionally the P3-HB comprises a P3-HB powder,
wherein optionally the P3-HB or P3-HB powder comprises between about 0.05% to 5%, or 0.05% to 10%, or 0.1% to 3%, or 0.1% to 2% by volume, or about 0.05%, 1%, 2%, 3%, 4% or 5% by volume of the PHA composition;
the PHA composition further comprises a high melting point ester, wherein optionally the high melting ester comprises a fatty acid ester, and optionally the high melting point ester comprises a micronized high melting point ester, and optionally the high melting point is between about 25° C. and 75° C., or is above about 30° C., or above about 40° C., or above about 50° C., or above about 60° C., or optionally the ester has a melting point between about 50° C. and 80° C.; and/or
the PHA composition is formulated as or made into a dispersion in water, optionally formulated as or made into a dispersion from the fermentation broth or after micronization of a PHA homopolymer, optionally a purified PHA homopolymer, and optionally the PHA homopolymer comprises: P-3HB or a PHA copolymers, or one or more of 3-hydroxybutyrate (3HB) homopolymer, or a copolymer of 3-hydroxybutyrate (3HB) and 4-hydroxybutyrate (4HB), or a copolymer of 3-hydroxybutyrate (3HB) and 3-hydroxyvalerate (3HV), or a copolymer of 3-hydroxybutyrate (3HB) and a 3-hydroxyhexanoate (3H) polymer, and the high-melting point ester.

In alternative embodiments, provided are methods of forming a water-based dispersion of a copolymer of PHA comprising: generating or obtaining the copolymer of PHA as a slurry or broth after cell fermentation to biosynthesize the PHA, followed by removing of cell debris and/or contaminants, optionally removing of cell debris by a process comprising cell disruption and/or enzymatic or light alkaline water washing.

In alternative embodiments, provided are products of manufacture comprising: paper or cardboard; a cellulose fiber-comprising packaging material, a plastic, a paint, a coating, an ink, an adhesive, a device and/or a fiber comprising: a composition as provided or described herein, or a composite structure or product of manufacture as provided or described herein, or a PHA composition as provided or described herein.

In alternative embodiments of the products of manufacture as provided herein: the plastic is or comprises: a single use plastic or a long-lasting plastic or plastic application, an acrylic, a polyester, a silicone, a polyurethane or a halogenated plastic, a conductive plastic, and optionally the conductive plastic comprises a polyacetylene, a thermoplastic, and optionally the thermoplastic comprises polyethylene (PE), polypropylene (PP), polystyrene (PS) and/or polyvinyl chloride (PVC), an amorphous plastic, and optionally the amorphous plastic comprises a methyl methacrylate (PMMA), a crystalline plastic, and optionally the crystalline plastic comprises high-density polyethylene (HDPE), polybutylene terephthalate (PBT) and/or polyether ether ketone (PEEK), or any combination thereof.

In alternative embodiments of the products of manufacture as provided herein: the fiber is used or fabricated as a woven fiber or nonwoven fiber, and optionally the product of manufacture comprises a diaper, a tissue, a fabric or a coating, and optionally the coating is placed or fabricated on a solid, flexible or semi-solid substrate, and optionally the solid, flexible or semi-solid substrate comprises a paper or a paperboard product.

In alternative embodiments of the products of manufacture as provided herein: the device is a medical device, optionally a medical device for in vivo use, and optionally the medical device is an implant, a pin, a rod, a substrate for cells, an artificial organ, a suture or a mesh, and optionally the composition or the composite structure acts or is a coating on the medical device.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

FIG. 1 is a schematic illustration of an exemplary PHA production process as provided herein, including fermentation of a microorganism to biosynthesize PHA (including use of micronutrients and oxygen infused or added to the fermentation culture); followed by separation of the PHA from the microorganism and the culture media; followed by suspension of the PHA in water or aqueous buffer; followed by isolation and/or purification of the PHA by for example, centrifugation and/or filtration; followed by spray drying of the PHA to generate a PHA powder.

FIG. 2 is a schematic flowchart of an exemplary process as provided herein illustrating a PHA dispersion process starting with PHA pellets or powder, where PHA pellets and/or powders are added to a micronizer such as a polymer jet micronizer, and the micronized PHA powder generated is mixed with an additive or additives as a PHA suspension in water at about between 50% to 60% PHA, which is then mixed in for example a high shear mixer to create a PHA dispersion, and this product can be packaged into containers such as “totes”, as described in further detail, below.

FIG. 3 is a schematic illustration of an exemplary process as provided herein for forming a PHA dispersion starting from a fermentation broth as described for example in FIG. 1C, the dispersion comprising a PHA suspension in water at about between 50% to 60% PHA, then subjected to mixing as a high shear mixing, this generating a PHA dispersion, and this product can be packaged into containers such as “totes”, as described in further detail, below.

FIG. 4 is a schematic illustration of an exemplary process as provided herein comprising preparing PHA pellets or powders (as described for example in FIG. 1), to which an additive or additives are added, to generate a product subjected to melt extrusion, then pelletization, where the PHA pellets can be packaged in containers or bags such as paper bags, and this product can be used as a melt laminating surface coating onto paper or paperboard, or for fertilizer, as described in further detail, below.

FIG. 5 is a schematic illustration of an exemplary process as provided herein comprising adding solid additives to a PHA suspension in water (see FIG. 1C), then subjecting this mixture to a mixer such as a high shear mixer or equivalent, then removing water in for example a centrifuge, then spray drying to generate a PHA powder, to which a liquid additive or additives is/are added and a melt extrusion is generated to fabricate PHA pellets, which can be packaged in bags such as paper bags or other containers for further fabrication, for example, for melt coating and/or laminating, as described in further detail, below.

The drawings set forth herein are illustrative of exemplary embodiments provided herein and are not meant to limit the scope of the invention as encompassed by the claims.

Like reference symbols in the various drawings indicate like elements.

### DETAILED DESCRIPTION

In alternative embodiments, provided are compositions, including products of manufacture and kits, comprising a polyhydroxyalkanoate (PHA) polymer. In alternative embodiments, PHA polymer comprises one or more PHA monomers. For example, in alternative embodiments, the PHA polymer comprises one or more of: a) 3-hydroxybutyrate, b) 4-hydroxybutyrate, c) 3-hydroxyvalerate, d) 3-hydroxyhexanoate, e) 3-hydroxypeptanoate, f) 3-hydroxyoctanoate, g) 3-hydroxydecanoate, h) 3-hydroxynonenoate or i) 3-hydroxy undecanoate.

In an exemplary embodiment, a PHA copolymer as provided herein comprises 3-hydroxybutyrate (3HB) and 4-hydroxybutyrate (4HB) in ratios ranging about 20:1 (95%/5%) to 1:1 (50%/50%). For example, 3HB and 4HB may be present at ratios of 3HB:4HB including 20:1, 15:1, 10:1, 9:1, 8:1, 7:1, 6:1, 5:1, 4:1, 3:1, 2:1 and 1:1. Ratios of 3HB:4HB of 6:1 (84%/16%) to 3:1 (75%/25%) have been tested and found to be suitable for use in compositions as described herein. Additionally, PHA copolymers comprising 3-hydroxybutyrate and any of the above mentioned comonomers are suitable for use in compositions as described herein.

A person having ordinary skill in the art will understand that PHA monomers having similar morphology to those described herein are suitable for use in a composition or product of manufacture as described herein. In alternative embodiments, exemplary monomers with similar morphology includes those with a property that lowers crystallinity, as measured by the standard method using a Differential Scanning Calorimeter (DSC), by between about 1% to 15%, or between about 0.5% to 20%, at the same co-monomer ratios due to a bulky monomer side chain.

In alternative embodiments, PHA copolymers used in compositions as provided herein, and methods for making them, and PHA copolymers used in methods as provided herein, have a number average molecular weight (Mn) (MW), as measured for example by Gel Permeation Chromatography (GPC), of between about 20,000 to 1,000,000, or between about 50,000 to 900,000, or between about 80,000 to 650,000, or between about 120,000 to 400,000.

Polydispersity of the exemplary PHA copolymers (for example, PHA copolymers used in compositions as provided herein, or PHA copolymers used in methods as provided herein,) can range from between about 1.6 to 2.8, to between about 1.7 to 2.5, or between about 1.8 to 2.4, or between about 1.4 to 2.8.

In alternative embodiments, PHA compositions as provided herein have properties and features that make them useful as replacements for environmentally harmful plastics, paints, coatings, inks, and adhesives. Exemplary uses include plastics such as single use plastics, or more long-lasting plastic applications, or in or as a coating for a fiber for use in nonwoven materials such as in diapers, tissue, fabrics, and coatings on paper, cardboard, paperboard products and cellulose fiber-comprising packaging materials. The formulation of the composition can be varied or tuned depending on the desired end use.

In alternative embodiments, different combinations of PHA monomers are used to give different mechanical, thermal, and other end use properties. In alternative embodiments, polymer properties that are designed and varied include viscosity, melt flow, crystallinity, film-forming and dispersion characteristics of the polymer. In an exemplary embodiment, it may be desirable for a PHA copolymer for use in a coating application to have a lower melt flow viscosity such as lower than about 40 Poise (Pascal) seconds (Pa sec, or Pa s) as measured by Brookfield cone and plate viscometer, and a less rigid film as this combination of features may provide a better barrier coating for paper stock that is flexible and typically bent during converting or usage, for example, to hold food items.

PHAs can be produced by bacteria such as, Paraburkholderia fungorum DSM 1749 or Azotobacter vinelandii growing on carbohydrate substrates, or Cupriavidus necator DSM 545 on fatty acid or carbohydrate substrates, or Methylosistis parvus OBBP growing on methane. Many of these microbes have been cultured to produce PHA on an industrial scale since the 1980's.

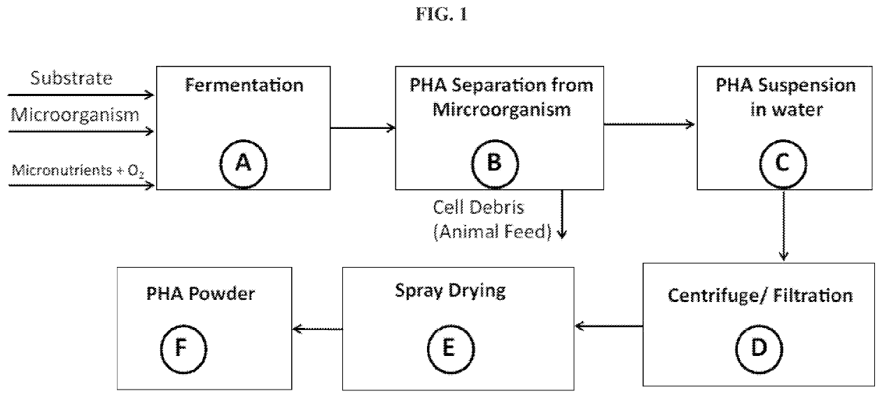

An exemplary (industrial) PHA production process used to make PHA used in compositions and methods as provided herein comprises use of a fermenter or equivalent to which a microorganism (for example, a bacterial culture) is added, and then fed substrates (for example, micronutrients) such as carbohydrates, fatty acids, nutrients and oxygen. After cell (microorganism) growth has taken place, nutrient types and quantities are adjusted for the microbes (microorganisms) to produce and accumulate PHA. In alternative embodiments, the microorganisms accumulate more than about 80% of their cell weight as PHA. The end point of the fermentation is determined by PHA accumulation rates, which can be monitored online. At the end of fermentation, microbes are lysed, for example, the fermentation broth is subjected to high temperatures to lyse the microbes; followed by total water reduction using for example, filtration and/or centrifugation. The PHA is then removed using for example, a cell separation and/or an extraction process. In alternative embodiments, any one or combination of three main types of separation processes that are commercially practiced can be used: solvent based, alkaline water based, and/or enzymatic. After the removal of the cell debris (which can be used as animal feed), a slurry is generated, and the slurry contains water and PHA granules (50 nanometers to several microns), which is then further subjected to filtration and/or centrifugation or equivalents to remove excess water; followed by spray drying or equivalent to obtain a PHA powder. FIG. 1 is a schematic illustration of an exemplary PHA production process as provided herein.

In addition to the above described PHA copolymers, in alternative embodiments the PHA composition as provided herein further comprises a viscosity modifying agent. The viscosity modifying agent may comprise an ester. For example, the ester may be the product of a fatty acid and a polyhydroxy alcohol. Exemplary fatty acid esters comprise lauric acid (C12), myristic acid (C14), palmitic acid (C16), stearic acid (C18), arachidic acid (C20), or behenic acid (C22). Exemplary fatty acid esters may have a melting point above 40° C., or above 50° C., or above 60° C.

Fatty acid esters of polyhydroxy compounds were tested and were found to lessen viscosity of exemplary PHA compositions. Some of the fatty acid esters weakened the films formed by the composition worse than others. Suitable polyhydroxy alcohol compounds can include one or more of glycerin, trimethyl propanol (TMP), pentaerythritol, sorbitol or mixtures thereof.

Moreover, in testing, unsaturated fatty acid esters did not significantly change the appearance and surface characteristics of films formed of exemplary PHA compositions at relatively low concentrations of less than about 10%, optionally ranging from between about 0.5 to 10.0%, or from between about 2% to 8%, or between about 5.0% to 12.0%. However, at higher concentrations greater than 10% or ranging from 10% to 20% or 40% or greater, undesirable effects are seen in the films, such as weakened film strength, haziness of the film preventing light from passing through, and incompatibility of the ester with the PHA co-polymer.

Advantageously, certain saturated fatty acid polyhydroxy esters mix well with the PHA copolymer, even up to 15% by weight. In alternative embodiments, films comprising PHA compositions having ester present in a range of about 10% have improved properties for machine applied coatings such as reduced viscosity, better penetration into the substrate, faster tack free times, and maintain water and oil resistance, which are useful for application to paper, cardboard or paperboard substrates and cellulose fiber-comprising packaging materials.

In addition, embodiments of coating films produced using the PHA composition have oil and water hold out in dry film thicknesses useful for coated substrates, ranging from between about 5 to 200 microns, from between about 10 to 100 microns, or between about 20 and 50 microns. In alternative embodiments there is a need to apply more than one coating layer on to paper, cardboard, paperboard and/or cellulose fiber-comprising packaging materials to be able to achieve the right film thickness to get the desirable water, oil and grease barrier properties. Therefore, the film thickness ranges listed above represent the total coating film thickness. Such thicknesses correspond to about 1, 2, 3, 4 or 5 g/m² and up to about 350, 400, 440 or 500 g/m² coating weight, or between about 10 to 350 g/m², or from between about 20 to 320 g/m². Advantageously, a lower coating thickness with an exemplary copolymer allows for a higher bend angle of the coated paper, cardboard or cellulose fiber-comprising packaging material without breaking surface integrity of the coating, which is a problem with pure PHA polymer coatings.

The viscosity of an exemplary PHA copolymer was measured with a Brookfield cone and plate rotational viscometer (Cap 2000+) using a #4 spindle at 10 revolutions per minute (rpm). This is a commonly used measurement in the paper industry to determine a single value low-shear viscosity of formulated coatings, like in the Technical Association of the Pulp and Paper Industry (TAPPI) Standard T-648 (“Viscosity of coating clay slurry”). This viscosity measurement is used to not only formulate paper coatings, but also determine their ability to be processed in coaters and laminators. Results of the measurements with the PHA copolymer for coating films are shown in Table 1. The pure PHA copolymer, without any additives, exhibited a viscosity value of 65.9 Poise (Pascal seconds, Pa s) at 200° C., but measurements went beyond the scale of the viscometer's dial when tested at lower temperatures as viscosity values were too high to measure with the set viscometer conditions. When the PHA copolymer was combined with 10% ester, viscosity measured using the same spindle and rotational speed (rpm) was unexpectedly lower as shown in the viscosity values in Table 1:

TABLE 1

| | | Viscosity of PHA |
|---|---|---|
| | Viscosity with additives | Copolymer w/o additives |
| Temperature (° C.) | (Poise, Pa s) | (Poise, Pa s) |
| 200 | 18.3 | 65.9 |
| 175 | 39.7 | Out of scale range |
| 170 | 42.1 | Out of scale range |
| 160 | 53.1 | Out of scale range |
| 150 | 67 | Out of scale range |

In addition to viscosity reduction, with expected melt flow improvement, the flexibility of a fully solidified (crystallized) film formed with exemplary embodiments of the PHA composition is also improved. When a film is prepared using 100% PHA copolymer, the fully solidified (crystallized) version can only be bent at an angle of about 15° before rupture of the film occurred. This result is observed with a film thickness greater than 5 mm.

At lower thicknesses, the bend angle increases. However, using the same thickness of 5 mils and a coating of 90% PHA copolymer with 10% ester, the bend angle increases to over 90°. This is a surprising effect that provides flexibility to packaging, a desirable end use feature.

In alternative embodiments, a 1 mm to 5 mm or a greater (for example, to about 10, 14 or 20 or more mm) film thickness is used to obtain sufficient barrier properties from a coating. As such, flexibility of a polymer film is of great importance, especially when higher thicknesses are required for barrier properties. In the situation of coatings that only require short term water or liquid hold out, thicknesses below 5 mm (for example, 0.5, 1, 2, 3, 4 mm) may be acceptable. If longer term barriers to moisture or oxygen are needed, coatings having a thickness greater than 5 mm may be required.

Additionally, the PHA composition having saturated fatty acid esters as provided herein can advantageously reduce production time and costs for coating substrates with films comprising the PHA composition. Production time and costs improve by reducing solidification time for films comprising the PHA composition. Additional lab testing showed that the addition of saturated fatty acid esters to a PHA copolymer, which was then used to form a PHA polymeric melt, provided an unexpected benefit to the PHA polymeric melt when it was coated on to a substrate and a continuous film was formed. In addition to lowering the melt viscosity without degrading the melt, the additive induces relatively rapid solidification of the film surface, allowing it to become tack free, a desirable feature. This is necessary when applying coatings on a machine where the substrate is rapidly rolled up for further processing. If at the point of contact with another surface (such as what happens on a paper machine) the coating is still in a tacky (non-crystalline) state, then it will adhere to that surface forming a laminate. Although the copolymers appear to have these properties that crystallize slowly making them more suitable for laminating than coating, most paper requires a surface coating as a barrier. In this case a non-tack surface is required within seconds as modern paper coating machines move at very fast speeds for economic reasons. Pure homopolymers of PHA's crystallize very fast and have high melt points. The copolymers have lower melt points but require time to fully crystallize once heat is removed. What occurs is a coating with a tacky surface that becomes non-tacky with time.

Polymeric materials can exist as solids without crystallization. In some cases, crystallization may take place over a very long time. Generally, the higher the molecular weight the slower crystallization can occur, but it is also dependent on the degree of crystallization or the ease of crystallization. If the areas that crystallize are abundant in the polymer, it will crystallize more rapidly. For example, after removal of heat, crystallization of various copolymers can take place within about 1 minute to about 120 minutes (min), and in alternative embodiments crystallization times for methods as provided herein are, for example, 1, 5, 10, 15, 20, 25, 30, 35, 40, 45, 50, 55, 60, 65, 70, 75, 80, 85, 90, 95, 100, 105, 110, 115, 120 min, and longer, for example, up to several days, for example, 1, 2, 3, 4, 5 or 6 days.

Transition from a glass to a rubber-like state of a polymer upon heating, and visa versa upon cooling, is an inherent polymer property called the Glass Transition Temperature or, T_{g}. The T_{g} is an important feature of polymer behavior, marking the region of changes in the physical film properties, such as hardness and elasticity. The T_{g}, determines polymer film properties such as hardness, elongation to break and Young's modulus, therefore characterizing the polymer.

For a PHA composition as provided herein, a characteristic transition occurs between the polymer's glass phase and the melting temperature of the modified polymer. An increased level of crystallinity and its evolution analogous to the behavior of conventional semi-crystalline polymers takes place. Without being bound by theory, or without being limited by any particular mechanism of action, it is believed that hydrophobic long-chain fatty acid ester molecules in the viscosity modifying agent of the PHA composition repel from hydrophilic entities, such as the cellulosic paper surface and the PHA molecules, therefore accelerating film formation with subsequent rapid transition of the tacky melt to non-tacky film. The feature of relatively rapid crystallization allows a paper, cardboard or paperboard substrate coated with the PHA composition to be wound up sooner than would be possible without the rapid solidification of the PHA composition film on the substrate. In contrast, a film comprising a pure, unmodified PHA copolymer solidifies more slowly, requiring more than twelve hours to reach a non-tacky state on a substrate surface. The property of relatively fast solidification (crystallization) beneficially reduces processing time and makes processing of paper, cardboard and paperboard stock coated with the PHA composition easier, faster, and thus, less costly.

Previously, annealing of the PHA film has been used to improve flexibility; however, as described above, the copolymer alone (without additives) crystallizes at such a slow rate that the time factor is prohibitive in a more modern paper or paperboard manufacturing facility. Thus, heating and cooling annealing processes are too costly both in energy and time.

In addition to the PHA compositions as provided herein comprising a viscosity modifying agent, the PHA composition may further comprise a nucleation agent. A person having ordinary skills in the art will understand that rapid nucleation forms smaller crystal structures which enable more flexibility when fully crystallized. Moreover, in testing, a PHA composition that does not include a nucleation agent takes about 18 hours to fully crystallize after solidification. A nucleation agent promotes smaller crystal structure or formation of crystallites as opposed to large spherulitic crystals within the PHA copolymer, which can give a film formed with the PHA composition including the nucleation agent additional flexibility. Exemplary nucleation agents include, without limitation, precipitated calcium carbonate, precipitated or fumed silica, talc, bentonite or montmorillonite clay, calcium sulfate, and boron nitride.

Interestingly, it was determined that a highly crystalline PHA, such as P3-HB (poly-3-hydroxy butyrate), can be used in a PHA composition as a nucleating agent when the PHA composition is in the form of a dispersion. It was found that addition of P3-HB in powder form (for example, optionally added at amounts of less than about 1000 microns, or less than about 100 microns, or less than about 40 microns, or between about 20 microns and 1000 microns) enabled more of the PHA polymer to be added to the PHA composition. For example, 0.005% to 1% of the PHA polymer could be added onto the surface after coating and while the coating was still in the melt stage, to rapidly crystallize the coating for further processing. Additionally, more substantial amounts (greater than 1% and up to 50%) could be added to the copolymer and dissolved prior to coating. This allows more variations in copolymer to polymer ratios while still preserving the end-of-life properties of compost ability and/or biodegradability.

In alternative embodiments, highly crystalline P3HB is added as a nucleating agent in a PHA composition in the form of a polymer melt. However, in a melt, the fully crystalline PHA (P3HB) dissolved in the less crystalline copolymer relatively rapidly. In contrast, in a dispersion where the fully crystallized P3HB was added to the dispersion at room temperature, and the dispersion was heated for only a short time to coalesce the particles, the pure P3HB maintained its crystalline state and acted as a good nucleation agent.

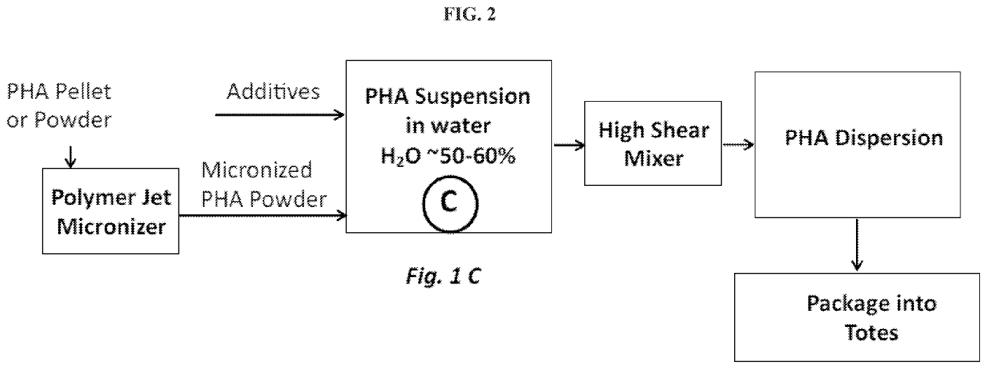

The PHA composition can be manufactured in the form a dispersion. A PHA composition in dispersion form (hereinafter PHA dispersion) may be produced according to the process shown in FIG. 2. In this method of producing a PHA dispersion, the starting material is a PHA pellet or a PHA powder. The pellets or powder can be micronized into small particles of about 50-500 nanometers, using, for example, a Jet Micronizer. The micronized PHA material can be added to a slurry tank along with water. Additives, such as those discussed above, can be added to the slurry tank prior to the micronized PHA and the entire mixture can be agitated under high shear conditions to disperse the micronized PHA powder into a dispersion. FIG. 2 is a schematic flowchart illustrating the PHA dispersion process that starts with PHA pellets or powder.

FIG. 2 illustrates an exemplary method for producing a PHA dispersion for coating from solid PHA.

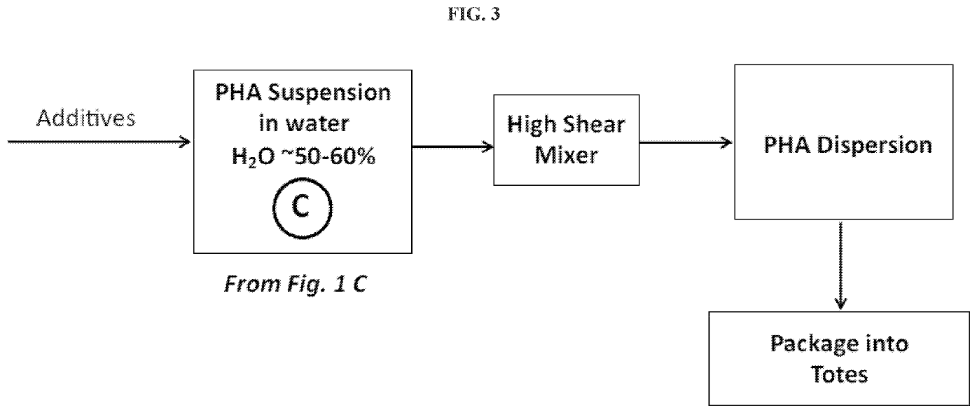

Another approach to producing a PHA dispersion is to start with fermentation broth of the PHA Copolymer from the PHA production process as shown in FIG. 1. After the broth has been cleaned of all organic/cell matter the copolymer particles remain in a submicron state. Saturated fatty acid polyhydroxy ester(s) and P3HB particles can be added to the fermentation broth and agitated appropriately to create the PHA dispersion. This process is shown in FIG. 3, which is a schematic illustration of the process for forming a PHA dispersion starting from the fermentation broth in FIG. 1 This method provides several desirable results such as less handling of the PHA polymer thereby lowering the overall cost of producing the PHA dispersion. In addition, the process produces smaller polymer particle sizes in the dispersion, and the PHA particles are completely amorphous. Therefore, the particles and other additives, especially the nucleation agent can coalesce quicker and form a uniform film on a substrate (such as paper or cardboard) in comparison to PHA dispersions produced from solid PHA that has already gone through one heat cycle and therefore has some crystallinity in it. Given the smaller polymer particle size, dispersing other ingredients or additives in the mixture is easier due to the micro turbulence occurring in the flow of the particles during coalescence.

In alternative embodiment, dispersions of PHA-comprising polymers as provided herein are used in water-based paper (or any cellulose fiber-comprising packaging material) barrier coating applications. Aqueous polymer dispersions offer advantages over extruded films formed from polymer melts. Aqueous polymer dispersions enable a relatively thin coating of the polymer to be applied on a substrate compared to melt polymer lamination processes with extrusion. Water acts as an excellent dispersing medium for the polymer, enabling a reduced viscosity of the dispersion relative to the polymer melt itself. Additionally, water-based dispersions have a relatively lower concentration of polymer, allowing for a thinner and more even coating to be applied at faster coating/machine speeds. Thus, improving the economics of the coating process.

Water-based PHA dispersions as provided herein can be applied using existing paper-coating equipment or printing press equipment. Advantageously, water based PHA dispersion coatings provide moisture, gas, and oil and grease barrier properties to paper, cardboard or paperboard. Moreover, packaging materials made from cellulose fiber and coated with PHA can be recycled and are biodegradable in soil, fresh water, or marine environments.

Thus, in alternative embodiments, paper, cardboard or paperboard packaging coated with PHA from a PHA dispersion as provided herein advantageously have barrier properties and are environmentally beneficial, which is particularly important for single-use foodservice packaging items, such as hot and cold beverage paper cups, plates, carry-out clamshells, bowls, paper wraps, etc. Fiber-based substrates coated with fossil-based plastic coatings cannot be easily recycled and cannot be composted within a practically reasonable time period. Thus, fossil-based plastic coatings are the primary reason for reduced recyclability of fiber-based materials and their lack of composting. In contrast, the combination of fiber-based materials coated with PHA as provided herein can be a major contributor to reducing plastics pollution and increasing recycling.

Choosing a PHA having a molecular weight and level of crystallinity, or ratio of crystalline to amorphous phases, that are suitable for relevant applications is important when making a PHA polymer dispersion for use as a coating. Additionally, viscosity of the polymer dispersion should be considered for processing on existing equipment during the substrate coating process. In alternative embodiments, the PHA dispersion viscosity can be from about a few centipoises (cps=1/1000 of Poise or mPa s) to several hundred centipoises. The viscosity target depends on the selected paper coating application method.

In alternative embodiments, provided are manufacturing methods for producing polymer dispersions, which methods can comprise any protocol or process known in the art. In alternative embodiments, methods that rely on active end groups, such as carboxylic termination, that can be further neutralized with base, are used in processes as provided herein. Alone PHAs have little surface functionality. Thus, exemplary methods for forming PHA dispersions as provided herein comprise use of additives or processing techniques to alter the continuous water phase to keep the PHA granules separated and dispersed until the dispersion is used. These exemplary techniques are similar to pigment dispersion techniques and can employ the use of dispersants and thickeners to prevent settling and agglomeration of PHA granules.

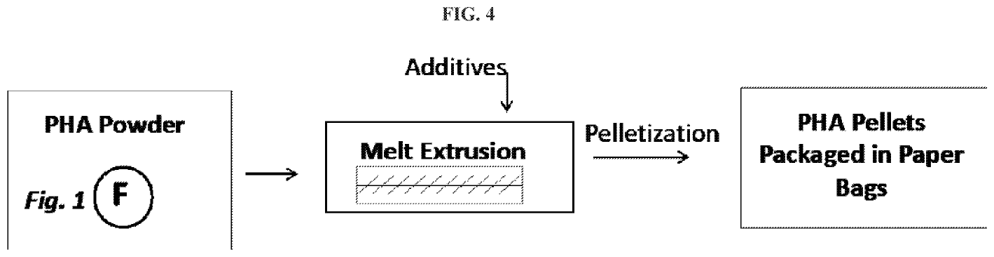

Attempts have been made to create coatings on paper/cellulose fiber via melt lamination and aqueous dispersions of PHA. In alternative embodiments, PHA polymers and additives, such as dispersing or emulsifying agents, anti-blocking agents, heat stabilizers, nucleating agents, and antioxidants, are used for producing effective coatings using PHA dispersions. A PHA polymer can provide a barrier to transmission of oxygen, moisture, and oil, while the other additives provide features to the polymer dispersion either during processing (dispersing/emulsifying agents, anti-blocking agents, nucleating agents) or during use (heat stabilizers, nucleating agents and antioxidants). In addition, in alternative embodiments, it is desirable for the coating to be suitable for use when coated paper, cardboard and paperboard are formed into packaging, which requires properties, such as, anti-blocking, heat sealing and resistance to crack at a fold and at a crease. FIG. 4 provides a schematic illustration of an exemplary process for preparing PHA pellets or powders that can be used for melt laminating surface coatings onto paper or paperboard.

In alternative embodiments, referencing the PHA production process shown in FIG. 1, once the PHA powder has been obtained via spray drying, the PHA powder can be put through a twin screw or single screw extruder to melt mix relevant additives and to form pellets of PHA mixed with additives, which can be used for extrusion coating of paper or extruded into a film that can be laminated onto paper.

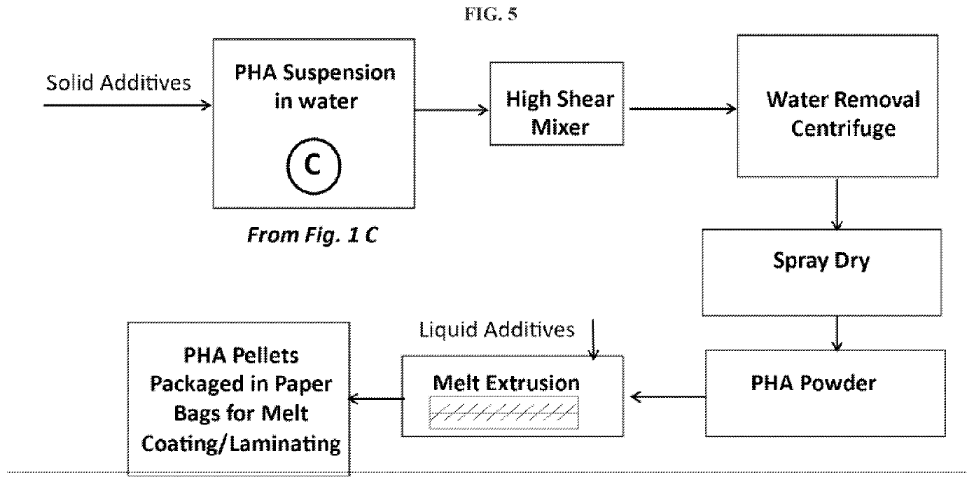

In alternative embodiments, aspects of the aqueous dispersion method and melt extrusion method can be combined in a hybrid process to produce solid PHA granule/powder that can be laminated as a coating under melted conditions, as described for example, in FIG. 5. This exemplary method can begin at FIG. 1, Box C. Solid additives can be introduced to the PHA slurry in water. The slurry can be agitated intensely until it turns into a PHA dispersion. The water can then be removed from the dispersion, and the remaining solids spray dried into a powder. Solid additives can then be added to the spray-dried powder and the mixture processed through a melt extruder into PHA pellets, which are, subsequently, packaged, for example, into paper bags, for shipment to an end user, who can melt the PHA pellets and use them as a laminated coating for a substrate of choice.

In alternative embodiments, antioxidants are used (are added to PHA mixtures) to prevent degradation of the polymer under high temperatures used to dry the continuous water phase and allow coalescence of the PHA particles. In alternative embodiments, exemplary antioxidants include, without limitation, sorbic acid, butylated hydroxytoluene (BHT) or butylated hydroxyanisole (BHA).

In alternative embodiments, antimicrobials are added to prevent the microbes that are ubiquitous in the environment from degrading the PHA dispersion before it is used. In alternative embodiments, the antimicrobials used are mild and usually decompose during production processing, typically, the dispersion to article processing. Exemplary antimicrobials include, without limitation, iodine, povidine, BHT (butylated hydroxytoluene), butylated hydroxyanisole (BHA), benzoic acid or ascorbic acid. In alternative embodiments, dispersants can be added to prevent or aid in preventing coagulation of the PHA particles. Exemplary dispersants may include, without limitation, esters of unsaturated acids, or polyacrylic acid.

In alternative embodiments, thickeners or viscosity modifiers are added to keep the continuous water phase viscous enough to prevent settling of the PHA particles. Exemplary viscosity modifiers comprise without limitation, alginates, or polyvinyl alcohol.

In alternative embodiments, ultraviolet light inhibitors are added if the article to be coated is intended to be used outside in direct sunlight. Exemplary UV light inhibitors may include, without limitation, Hindered Amine Light stabilizers, stearates of magnesium (Mg) or Mg oxides, or zinc (Zn) or Zn oxides.

In alternative embodiments, stabilizers are added to the composition as well. Exemplary stabilizers may include, without limitation, polyvinyl alcohol (PVOH), vinyl alcohol, soaps of fatty acids, pyrrolidone, and ethylene/propylene oxides.

Once the additives are sufficiently mixed, the slurry becomes a dispersion. Mixing times can vary depending on components. A person having ordinary skill in the art can determine suitable mixing times. The resulting dispersion can be packaged into containers, for example, totes for shipping the dispersion (FIG. 2 and FIG. 3) and paper/plastic bags for shipping the solid PHA pellets/powders (FIG. 4).

In addition to viscosity reduction, which is an indication of melt flow improvement, the flexibility of a fully solidified (crystallized) film formed with exemplary embodiments of the PHA composition was also improved. When a film was prepared using 100% PHA copolymer, the fully solidified (crystallized) version could only be bent at an angle of about 15 degrees before rupture of the film occurred. This result was observed with a film thickness greater than 5 mils. At lower thicknesses, the bend angle increased.

In alternative embodiments, about 5 mils (mls) or a greater (to about 10, 15, 20 or 25 mls) film thickness is generated to obtain sufficient barrier properties from a paper coating. Flexibility of a polymer film may be important, especially when higher thicknesses are required for barrier properties. In the situation of coatings that only require short term water or liquid hold out, thicknesses below 5 mils (for example, between about 0.5 and 5 mls) may be acceptable. However, if longer term barriers to moisture or oxygen are needed, coatings having a thickness greater than 5 mils may be required. In such situations, flexibility of the film is important. Testing showed that an exemplary PHA composition having glycerin mono stearate (GMS), in fully solidified (crystallized) film form, could be bent to about 900 even at a thickness above 5 mils. Increased film flexibility while maintaining moisture and oxygen barrier properties is desirable.

Additionally, the PHA composition having saturated fatty acid esters advantageously reduces production time and costs for coating substrates with films comprising the PHA composition. Production time and costs are improved by reducing solidification time for films comprising the PHA composition. Testing showed that the addition of saturated fatty acid esters to a PHA copolymer, which was then used to form a PHA polymeric melt, provided an unexpected benefit to the PHA polymeric melt when it was coated onto a substrate and a continuous film was formed. Fatty acid esters reduced melt viscosity, without degrading the melt, and induced relatively rapid solidification of the film (that is, within minutes), which was applied as a melt and dried onto the surface of the substrate (such as paper or cardboard). For example, solidification may take place within about 1 minute to about 120 minutes, including for example, 1 minute, 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, or 120 minutes. As used herein, the terms solidification and crystallization shall be used interchangeably and shall have the same meaning.

Regarding the crystallization process, after a polymeric melt is applied onto a substrate, film annealing, which is time and temperature dependent, proceeds. When an amorphous polymer is heated, the temperature at which the polymer structure turns “viscous liquid or rubbery” is called the Glass Transition Temperature, Tg. It can also be defined as a temperature at which the amorphous polymer takes on characteristic glassy-state properties like brittleness, stiffness and rigidity (upon cooling). Generally, amorphous polymers only exhibit a Tg, while crystalline polymers exhibit a Tm (melt temperature) and typically a Tg since there is usually an amorphous portion of the polymer as well (“semi”-crystalline). The value of Tg depends on the mobility of the polymer chain.

The transition from a glass to a rubber-like state is an important feature of polymer behavior, marking a region of changes in the physical properties, such as hardness and elasticity. At Tg, changes in hardness, volume, percent elongation to break and Young's modulus of solids can be seen.

For the PHA composition, a characteristic transition occurs between the polymer's glass transition and the melting temperature of the modified polymer. An increased level of crystallinity and crystalline evolution analogous to the behavior of conventional semi-crystalline polymers takes place. Without being bound by theory, it is believed that hydrophobic long-chain fatty acid ester molecules in the viscosity modifying agent of the PHA composition repel from hydrophilic entities, such as the cellulosic paper surface and the PHA molecules, therefore accelerating film formation, with subsequent rapid transition of the tacky melt to non-tacky film. The feature of relatively rapid crystallization allows a substrate coated with the PHA composition (such as a paper, cardboard or paperboard product) to be wound up sooner than would be possible without the rapid solidification of the PHA composition film on the substrate. In contrast, a film comprising a pure, unmodified PHA copolymer solidifies more slowly, requiring more than twelve hours to reach a non-tacky state on a substrate surface. The property of relatively fast solidification (crystallization) beneficially reduces processing time and makes processing of paper, cardboard and paperboard stock coated with the PHA composition easier, faster, and thus, less costly.

Previously, annealing of the PHA film has been used to improve flexibility; however, as described above, the copolymer alone (without additives) crystallizes at such a slow rate that the time factor is prohibitive in a more modern paper or paperboard manufacturing facility. Thus, heating and cooling annealing processes are too costly both in energy and time.

In addition to the PHA composition comprising a viscosity modifying agent, the PHA composition may further comprise a nucleation agent. A person having ordinary skill in the art will understand that rapid nucleation forms smaller crystal structures which enable more flexibility when fully crystallized. Moreover, in testing, a PHA composition that did not include a nucleation agent took about 18 hours to fully solidify and crystallize. A nucleation agent can promote smaller crystal structure or formation of crystallites as opposed to large spherulitic crystals within the PHA copolymer, which can give a film formed with the PHA composition including the nucleation agent additional flexibility. Exemplary nucleation agents include, without limitation, precipitated calcium carbonate, precipitated or fumed silica, talc, bentonite or montmorillonite clay, calcium sulfate, and boron nitride.

Products of Manufacture Coated with PHA Compositions

Provided are products of manufacture comprising: paper, paperboard or cardboard; a plastic, a paint, a coating, an ink, an adhesive, a device and/or a fiber comprising or coated with a PHA composition as provided herein, for example, a polyhydroxyalkanoate (PHA) polymer and a viscosity modifying agent.

In alternative embodiments, dispersions of polymers as provided herein are used in water-based paper coating applications. Aqueous polymer dispersions offer advantages over extruded films formed from polymer melts. Aqueous polymer dispersions enable a relatively thin coating of the polymer to be applied on a substrate compared to melt polymer lamination processes with extrusion. Water acts as an excellent dispersing medium for the polymer, enabling a reduced viscosity of the dispersion relative to the polymer melt itself. Additionally, water-based dispersions have a relatively lower concentration of polymer, allowing for a thinner and more even coating to be applied at faster coating/machine speeds, thus, improving the economics of the coating process. In addition, from an energy management perspective, the energy needed to evaporate excess water from the polymer dispersion after application is less than the energy required to cool an extruded film formed on a substrate from a polymer melt.

Water-based dispersions of PHA compositions as provided herein can be applied using existing paper-coating equipment or printing press equipment. Advantageously, water based PHA dispersion coatings provide moisture, gas, and oil and grease barrier properties to paper or paperboard. Moreover, packaging materials made from cellulose fiber and coated with PHA can be recycled and are biodegradable in soil, fresh water, or marine environments.

Thus, paper or paperboard packaging, or cellulose fiber-comprising packaging materials, coated with a PHA-comprising composition as provided herein from a PHA dispersion advantageously has barrier properties and is environmentally beneficial, which is particularly important for single-use foodservice packaging items, such as hot and cold beverage paper cups, plates, carry-out clamshells, bowls, paper wraps, etc. Fiber-based substrates coated with fossil-based plastic coatings cannot be recycled and cannot be composted. Thus, fossil-based plastic coatings are the primary reason for reduced recyclability of fiber-based materials and their lack of composting. In contrast, the combination of fiber-based materials coated with PHA can be a major contributor to reducing plastics pollution and increasing recycling.

Choosing a PHA having a molecular weight and level of crystallinity, or ratio of crystalline to amorphous phases, that are suitable for relevant applications is important when making a PHA polymer dispersion for use as a coating. Additionally, viscosity of the polymer dispersion should be considered for processing on existing equipment during the substrate coating process. In embodiments, the PHA dispersion viscosity can be from about a few centipoises (cps=1/1000 of Poise or mPa s) to several hundred centipoises. The viscosity target depends on the selected paper coating application method.

Numerous manufacturing methods are available for producing polymer dispersions as provided herein, and for practicing methods as provided herein. Some methods rely on active end groups, such as carboxylic termination, that can be further neutralized with base. Alone PHAs have little surface functionality. In alternative embodiments, methods for forming PHA dispersions use additives or processing techniques to alter the continuous water phase to keep the PHA granules separated and dispersed until the dispersion is used. The techniques are similar to pigment dispersion techniques and employ the use of dispersants and thickeners to prevent settling and agglomeration of PHA granules.

Attempts have been made to create coatings on paper/cellulose fiber via melt lamination and aqueous dispersions of PHA. Suitable PHA polymers and other additives, such as dispersing or emulsifying agents, antiblocking agents, heat stabilizers, nucleating agents, and antioxidants, are typically used for producing effective coatings using PHA dispersions. A PHA polymer can provide a barrier to transmission of oxygen, moisture, and oil, while the other additives provide features to the polymer dispersion either during processing (dispersing/emulsifying agents, antiblocking agents, nucleating agents) or during use (heat stabilizers, nucleating agents and antioxidants). In addition, in alternative embodiments, it is desirable for the coating to be suitable for use when coated paper and paperboard are formed into packaging, which requires properties, such as, anti-blocking, heat sealing and resistance to crack at a fold and at a crease. FIG. 4 provides a schematic illustration of preparing PHA pellets or powders that can be used for melt laminating surface coatings onto paper or paperboard.

Referencing the exemplary PHA composition production process shown in FIG. 1, once the PHA powder has been obtained via spray drying, the PHA powder can be put through a twin screw or single screw extruder to melt mix relevant additives and to form pellets of PHA mixed with additives, which can be used for extrusion coating of paper or extruded into a film that can be laminated onto paper.

In alternative embodiments, aspects of the aqueous dispersion method and melt extrusion method are combined in a hybrid process to produce solid PHA granule/powder that can be laminated as a coating under melted conditions. This method can begin at FIG. 1, Box C. Solid additives can be introduced to the PHA slurry in water. The slurry can be agitated intensely until it turns into a PHA dispersion. The water can then be removed from the dispersion, and the remaining solids spray dried into a powder. Solid additives can then be added to the spray-dried powder and the mixture processed through a melt extruder into PHA pellets, which are, subsequently, packaged, for example, into paper bags, for shipment to an end user, who can melt the PHA pellets and use them as a laminated coating for a substrate of choice.

Antioxidants can be used to prevent degradation of the polymer under high temperatures used to dry the continuous water phase and allow coalescence of the PHA particles. Exemplary antioxidants may include, without limitation, sorbic acid, butylated hydroxytoluene (BHT) or butylated hydroxyanisole (BHA).

Antimicrobials can be added to prevent the microbes that are ubiquitous in the environment from degrading the PHA dispersion before it is used. In embodiments, the antimicrobials used are mild and usually decompose during production processing, typically, the dispersion to article processing. Exemplary antimicrobials may include, without limitation, BHT, BHA, benzoic acid or ascorbic acid.

Dispersants can be added to prevent or aid in preventing coagulation of the PHA particles. Exemplary dispersants may include, without limitation, esters of unsaturated acids, or polyacrylic acid.

Thickeners or viscosity modifiers can be added to keep the continuous water phase viscous enough to prevent settling of the PHA particles. Exemplary viscosity modifiers may include, without limitation, alginates, or polyvinyl alcohol.

Ultraviolet light inhibitors can be added if the article to be coated is intended to be used outside in direct sunlight. Exemplary UV light inhibitors may include, without limitation, Hindered Amine Light stabilizers, stearates of magnesium or zinc.

Stabilizers can be added to the composition as well. Exemplary stabilizers may include, without limitation, PVOH, vinyl alcohol, soaps of fatty acids, pyrrolidone, and ethylene/propylene oxides.

Once the additives are sufficiently mixed, the slurry becomes a dispersion. Mixing times can vary depending on components. A person having ordinary skill in the art can determine suitable mixing times. The resulting dispersion can be packaged into containers, for example, totes for shipping the dispersion (FIG. 2 and FIG. 3) and paper/plastic bags for shipping the solid PHA pellets/powders (FIG. 4).

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

A number of embodiments of the invention have been described. Nevertheless, it can be understood that various modifications may be made without departing from the spirit and scope of the invention. Accordingly, other embodiments are within the scope of the following claims.

1-36. (canceled)

37. A method of forming a polyhydroxyalkanoate (PHA) copolymer, comprising:
generating a copolymer of PHA as a slurry or broth after cell fermentation to biosynthesize the PHA; and
removing any cell debris and/or contaminants.

38. The method of claim 37, including removing the cell debris by cell disruption and/or enzymatic or light alkaline water washing.

39. The method of claim 37, further comprising suspending the PHA in water to make a dispersion.

40. The method of claim 37, wherein the PHA copolymer comprises 3-hydroxybutyrate (3HB) homopolymer, a copolymer of 3-hydroxybutyrate (3HB) and 4-hydroxybutyrate (4HB), a copolymer of 3-hydroxybutyrate (3HB) and 3-hydroxyvalerate (3HV), or a copolymer of 3-hydroxybutyrate (3HB) and 3-hydroxyhexanoate (3HH) polymers.

41. A method of producing a polyhydroxyalkanoate (PHA) polymer, comprising:
culturing a PHA-producing microorganism, micronutrients, and oxygen in a vessel;
separating PHA from the cultured microorganism;
suspending the PHA in water;
generating a purified PHA from the PHA suspension by removing any remaining non-PHA debris from the microorganism.

42. The method of claim 41, wherein the vessel comprises a culture or fermentation reaction vessel.

43. The method of claim 41, wherein the separation uses filtration or centrifugation.

44. The method of claim 41, wherein the suspension uses purified or distilled water.

45. The method of claim 41, wherein generating the purified PHA comprises centrifuging or filtering.

46. A composition comprising a polyhydroxyalkanoate (PHA) polymer and up to 15 wt % of a viscosity modifying agent comprising a combination of:
an ester of lauric acid, myristic acid, palmitic acid, arachidic acid, or behenic acid; and
glycerin, trimethyl propanol (TMP), pentaerythritol, or combinations thereof.

47. The composition of claim 46, wherein the PHA polymer comprises a PHA copolymer and the PHA polymer or the PHA copolymer comprises 3-hydroxybutyrate (3HB) homopolymer, a copolymer of 3-hydroxybutyrate (3HB) and 4-hydroxybutyrate (4HB), a copolymer of 3-hydroxybutyrate (3HB) and 3-hydroxyvalerate (3HV), or a copolymer of 3-hydroxybutyrate (3HB) and 3-hydroxyhexanoate (3HH) polymers.

48. The composition of claim 47, wherein the PHA polymer or the PHA copolymer comprises 8% to 30% 4HB and 92% to 70% 3HB.

49. The composition of claim 48, wherein the PHA polymer comprises 3-hydroxybutyrate, 4-hydroxybutyrate, or a combination thereof.

50. The composition of claim 46, further comprising about 0.01% to about 5 vol % of a nucleating agent.

51. The composition of claim 46, having a non-tack coating time of less than about 30 seconds.

52. The composition of claim 46, further comprising:
an antioxidant comprising sorbic acid, butylated hydroxytoluene (BHT), butylated hydroxyanisole (BHA), or combination thereof,
an antimicrobial comprising BHT, BHA, benzoic acid, ascorbic acid or combination thereof;
a dispersant comprising an ester of an unsaturated acid, an ester of polyacrylic acid, or combination thereof,
an ultraviolet light inhibitor comprising a hindered amine light stabilizers, a stearate of magnesium or zinc, or combination thereof,
a stabilizer comprising polyvinyl alcohol (PVOH), vinyl alcohol, a soap of a fatty acid, pyrrolidone, an ethylene oxide, a propylene oxide, or combination thereof,
an emulsifying agent comprising a polyethylene oxide addict of a fatty acid and fatty alcohol, a polysorbate, or combination thereof; or
an anti-blocking agent comprising an inorganic mineral pigment, diatomaceous earth, talc, sodium or potassium aluminosilicate, a mineral mixture of sodium and potassium aluminosilicates, a silica, or combinations thereof.

53. A product of manufacture comprising paper, cardboard, a cellulose fiber-comprising packaging material, a plastic, a paint, a coating, an ink, an adhesive, a device, and/or a fiber containing the PHA copolymer made by the method of claim 1.

54. A product of manufacture comprising paper, cardboard, a cellulose fiber-comprising packaging material, a plastic, a paint, a coating, an ink, an adhesive, a device, and/or a fiber containing the the PHA polymer made by the method of claim 5.

55. A product of manufacture comprising paper, cardboard, a cellulose fiber-comprising packaging material, a plastic, a paint, a coating, an ink, an adhesive, a device, and/or a fiber containing the composition of claim 11.
